(19) United States
(12) Patent Application Publication
(10) Pub. No.: US 2008/0108906 A1
(43) Pub. Date: May 8, 2008

(54) Method for calculating and analyzing heart rhythm

(76) Inventor: Kuo-Yuan Chang, Shing-Tien City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/594,202

(22) Filed: Nov. 8, 2006

(51) Int. Cl. A61B 5/0402

(52) U.S. Cl. 600/515

(57) ABSTRACT

A method for calculating the heart rhythm includes: measuring a plurality of electrical heartbeat signals; evaluating the electrical heartbeat signals on the basis of a Fourier transformation to calculate a frequency spectrum of the electrical heartbeat signals, wherein at least a characteristic frequency presents within a frequency range of the frequency spectrum; and utilizing the characteristic frequency to get the heart rhythm. The method for analyzing the heart rhythm is to judge the number of the characteristic frequency within the frequency range to decide the frequency number of the heart rhythm.

### BACKGROUND OF THE INVENTION

1. Field of the Invention

The present invention relates to the method for calculating and analyzing the heart rhythm, and more especially, to the method for calculating and analyzing the heart rhythm by utilizing a frequency spectrum calculated by evaluating the electrical heartbeat signals on the basis of a Fourier transformation.

2. Background of the Related Art

The normal heart rhythm of the people is 60-100 times per minute and may be affected by many reasons, for example, the heart of a life beats faster when the life moves fast or take exercises, and the heart of the life beats slower when the life is rest or sleeps. Besides, the excited autonomic nerve, stimulative coffee or tea, fever, nervousness, pressure, pain, air hunger, anemia, medicine, pulmonary embolism or heart disease may make effects on the heart rhythm, such as arrhythmia which may means that the intervals of the heartbeats are different, or the heart rhythm is too fast or too slow.

In general, the heart rhythm is acquired by measuring the pulse within one minute by using an electric sphygmomanometer to get the number of times per minute. However, if the heartbeat is more than 240 times per minute or less than 30 times per minute, it is difficult to measure the number of times of the heartbeat by using the electric sphygmomanometer.

On the other hand, an electrocardiogram is also utilized to measure the heart condition. The electrocardiogram records the currents produced by the heartbeat, please refer FIG. 1, the electrocardiogram is composed of several PQRST waves. The intervals between every two neighboring heartbeats are calculated by measuring the internals between every two PQRST waves by a ruler, and then the intervals between every two heartbeats are utilized to judge whether the frequencies between every two heartbeats are clear or not. When the frequencies between every two heartbeats are different, it may be indicated the frequencies of the heart rhythm are not identical. However, it is time consuming to calculate the frequencies between every two heartbeats by measuring the internal between two PQRST waves, and it is also not convenient.

### SUMMARY OF THE INVENTION

In order to solve the forgoing problems, one object of this invention is to provide a method for calculating the heart rhythm. The characteristic of accurate heart rhythm is acquired by applying a Fourier transformation.

One object of this invention is to provide a method for analyzing heart rhythm. The accurate frequency of heart rhythm is acquired by measuring the main intensity of heart rhythm within a frequency range.

Accordingly, one embodiment of the present invention provides a method for calculating the heart rhythm, which includes: measuring a plurality of electrical heartbeat signals; evaluating the electrical heartbeat signals on the basis of a Fourier transformation to calculate a frequency spectrum of the electrical heartbeat signals, wherein at least a characteristic frequency presents within a frequency range of the frequency spectrum; and utilizing the characteristic frequency to get the heart rhythm.

Another embodiment of the present invention provides a method for analyzing the heart rhythm, which includes: measuring a plurality of electrical heartbeat signals; evaluating the electrical heartbeat signals on the basis of a Fourier transformation to calculate a frequency spectrum of the electrical heartbeat signals; and judging a number of a characteristic frequency within a frequency range of the frequency spectrum to decide the frequency number of the heart rhythm.

Another embodiment of the present invention provides a method for analyzing the heart rhythm, which includes: measuring a plurality of electrical heartbeat signals; evaluating the electrical heartbeat signals on the basis of a Fourier transformation to calculate a frequency spectrum of the electrical heartbeat signals; and counting a number of a characteristic frequency within a frequency range of the frequency spectrum to judge whether the heart rhythm is under a condition of arrhythmia.

### BRIEF DESCRIPTION OF THE DRAWINGS

FIG. 1 is a diagram illustrating a conventional electrocardiogram;

FIG. 2 is a flow chart illustrating the method for calculating the heart rhythm in accordance with an embodiment of the present invention;

FIG. 3a and FIG. 3b are diagrams recording the electrical heartbeat signals of a human being and its frequency spectrum, respectively;

FIG. 4a and FIG. 4b are other diagrams recording the electrical heartbeat signals of a human being and its frequency spectrum, respectively;

FIG. 5a and FIG. 5b are other diagrams recording the electrical heartbeat signals of a human being and its frequency spectrum, respectively;

FIG. 6a and FIG. 6b are other diagrams recording the electrical heartbeat signals of a human being and its frequency spectrum, respectively;

FIG. 7 is a flow chart illustrating the method for analyzing the heart rhythm in accordance with another embodiment of the present invention;

FIG. 8 is a frequency spectrum of a normal human being in accordance with the present invention;

FIG. 9a, FIG. 9b and FIG. 9c are diagrams recording the electrical heartbeat signals of a human being, its frequency spectrum and its electrocardiogram, respectively; and

FIG. 10a, FIG. 10b and FIG. 10c are diagrams recording the electrical heartbeat signals of a human being, its frequency spectrum and its electrocardiogram, respectively.

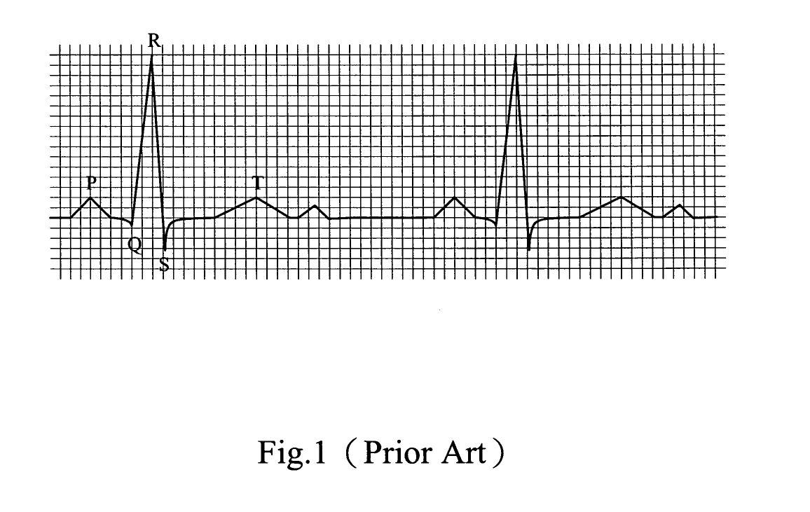

### DETAILED DESCRIPTION OF THE INVENTION

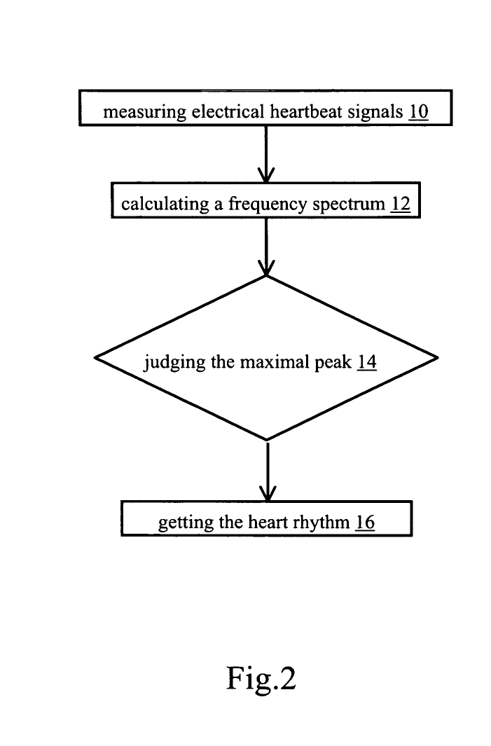

FIG. 2 is a flow chart illustrating the method for calculating the heart rhythm in accordance with one embodiment of the present invention. The method includes the following steps: measuring a plurality of electrical heartbeat signals of the beings (step 10) by a heart monitor or a physiological monitoring apparatus. Next, converting the electrical heartbeat signals into a frequency spectrum of the electrical heartbeat signals with a Fourier transformation (step 12). There are several peaks corresponding to several characteristic frequencies, respectively, in the frequency spectrum. Then, judging the maximal peak among the peaks within a designated frequency range of the frequency spectrum (step 14) and utilizing the maximal peak to get a corresponding principal characteristic frequency (P), which represents the heart rhythm of the beings (step 16). In one embodiment, the measurable region of the beings may be an arm, wrist, finger or an ankle. Moreover, the electrical heartbeat signals may be measured by infrared signals, thermal radiation signals, sound signals, current signals, voltage signals, magnetic induction signals or pressure signals.

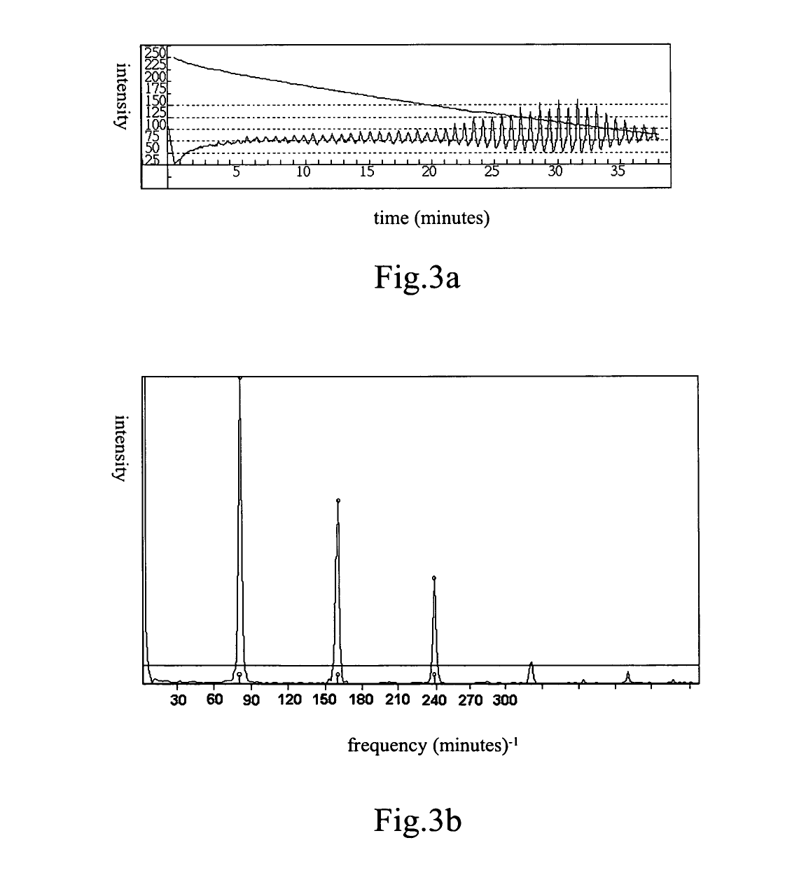

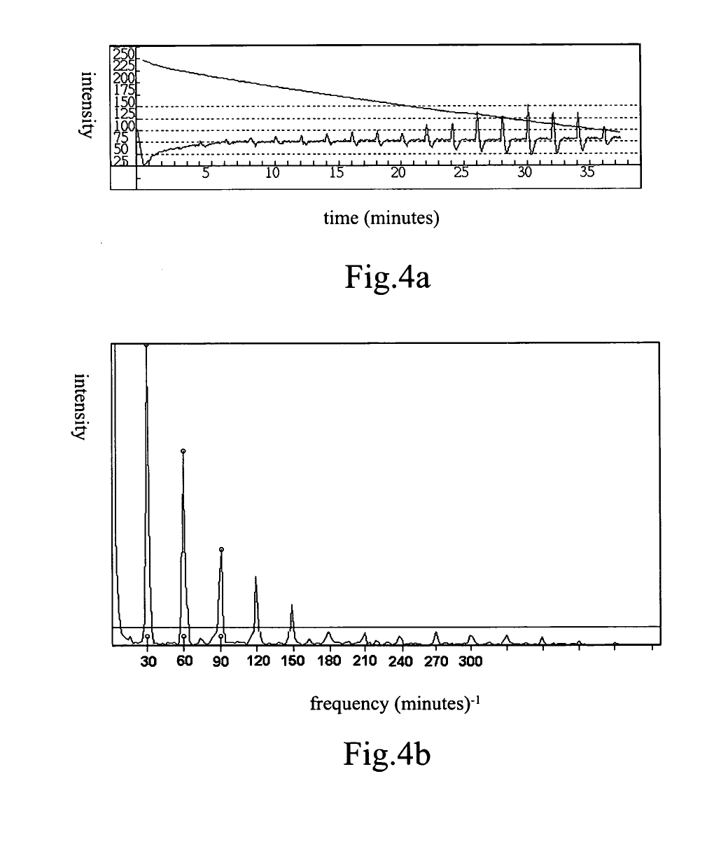

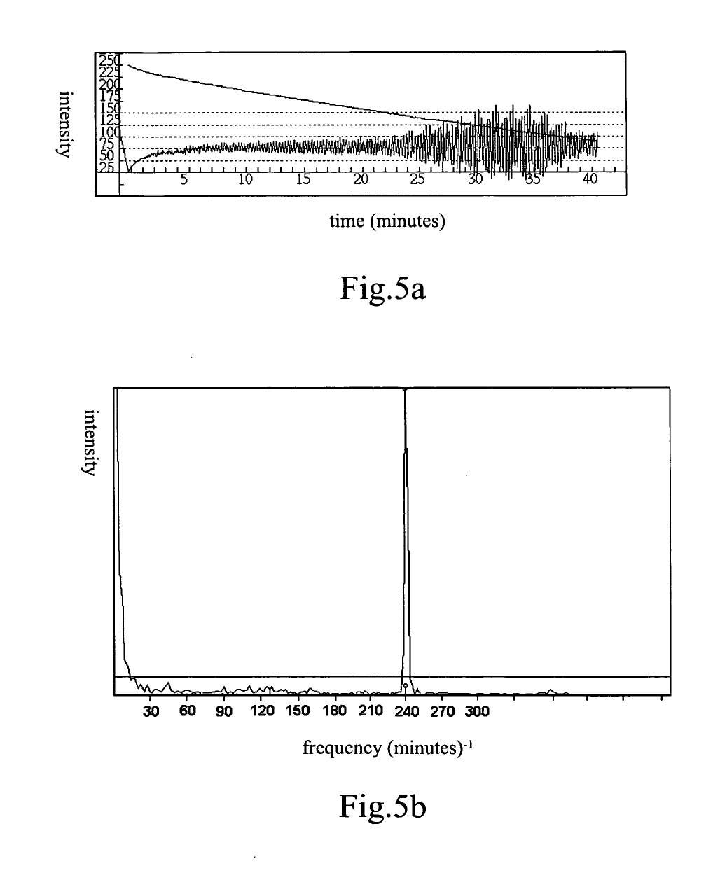

FIG. 3a is a diagram recording the electrical heartbeat signals in relation to an ongoing intensity of a human being. FIG. 3b is the frequency spectrum of the electrical heartbeat signals shown in FIG. 3a on the basis of a Fourier transformation in accordance with one embodiment of the present invention. Shown in FIG. 3b, several peaks are presented within the frequency spectrum and located on different frequencies. In the embodiment, the maximal peak is located on the principal characteristic frequency with in the frequency range of 0 to 90, such as 80 times per minute. Therefore, it is indicated that the heart rhythm of the human being is 80 times per minute. On the other hand, there is single peak located within other frequency range, such as 165 (minutes ⁻¹) and 240 (minutes ⁻¹), respectively. FIG. 4a and FIG. 5a are other different diagrams recording the electrical heartbeat signals in relation to ongoing intensities, and FIG. 4b and FIG. 5b are their frequency spectrums on the basis of a Fourier transformation, respectively. Shown in FIG. 4b and FIG. 5b, the maximal peaks are located on the principal characteristic frequency with 30 times per minute and 240 times per minute, respectively, therefore, the heart rhythm are 30 times per minute and 240 times per minute, respectively.

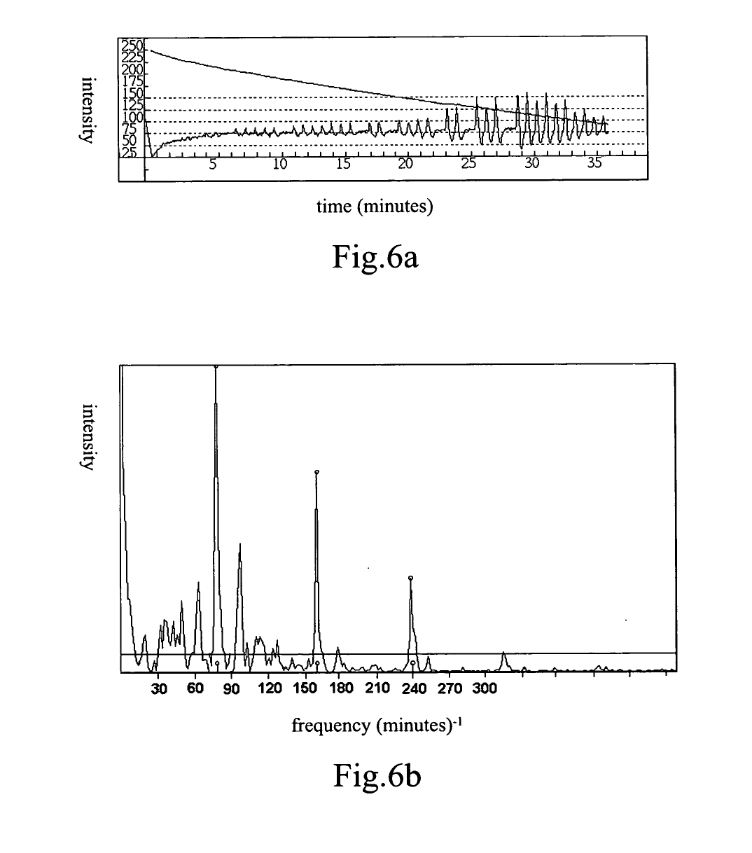

Continuously, the designated frequency range of the frequency spectrum is from zero to a 1.5 times of the heartbeat number per minute. For example, the frequency range is from 0 to 120 times per minute in accordance with FIG. 3b in which the heart rhythm is 80 times per minute. In general, shown in FIG. 3b, the frequency spectrum of a normal human should only present single peak corresponding one characteristic frequency (principal characteristic frequency) within the foregoing designated frequency range of the frequency spectrum. Please refer to FIG. 6a and FIG. 6b, they are other diagrams recording the electrical heartbeat signals and its frequency spectrum, respectively. Shown in FIG. 6b, if more than one peak presents within the designated frequency range, understandably, the peaks correspond to a principal characteristic frequency and other characteristic frequencies, wherein the principal characteristic frequency corresponds to the heart rhythm, accordingly. On the other hand, the designated frequency range of the frequency spectrum may be selected from the frequency of 0.84 P to the frequency of 1.16 P, wherein the P corresponds to the value of the principal characteristic frequency.

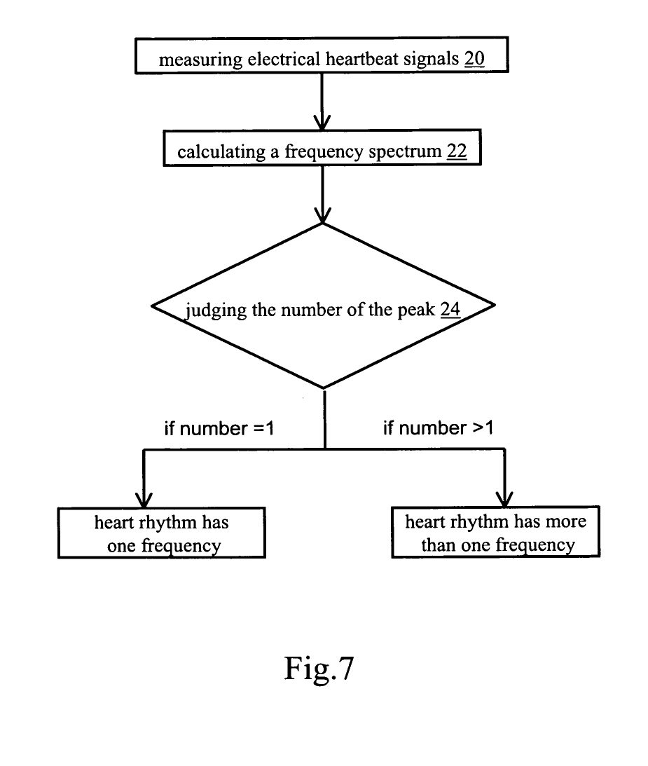

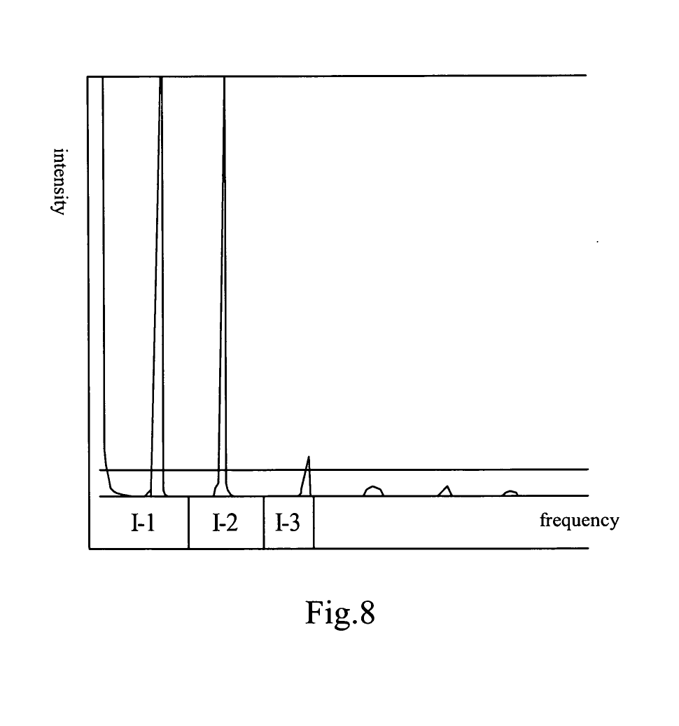

The present invention also provides a method for analyzing the heart rhythm, please refer to FIG. 7, it is a flow chart illustrating the method for analyzing the heart rhythm in accordance with one embodiment of the present invention. The method includes the following steps: measuring a plurality of electrical heartbeat signals of the beings (step 20) by a heart monitor or a physiological monitoring apparatus. Next, evaluating the electrical heartbeat signals on the basis of a Fourier transformation to calculate a frequency spectrum of the electrical heartbeat signals (step 22), and the frequency spectrum shown in FIG. 8 is divided into three frequency regions, as the first region (I-1), the second region (I-2) and the third region (I-3), wherein the frequency range of the first region is from zero to the 1.5 times of heartbeat number per minute. Then, judging the number of the peak within the first region to decide a frequency number of the heart rhythm (step 24). Understandably, if only single peak corresponding one characteristic frequency presents within the first region, it is dedicated that the heart rhythm has one frequency, and if more than one peak corresponding more than one characteristic frequency presents within the first region, it is indicated that the heart rhythm has more than one frequency.

Wherein, the frequency range of the second region is from 1.5 times of heartbeat number per minute to 3 times of heartbeat number per minute, and the frequency range of the third region is from 3 times of heartbeat number per minute to 4.5 times of heartbeat number per minute. For example, if the heartbeat number per minute is 80 times per minute, the frequency range of the first region is from 0 to 120 times per minute, the frequency range of the second region is 120 to 240 times per minute, and the frequency range of the third region is 240 to 360 times per minute. In general, within a frequency spectrum of a normal human being, shown in the FIG. 8, three different peaks will present within the first region, the second region and the third region, respectively.

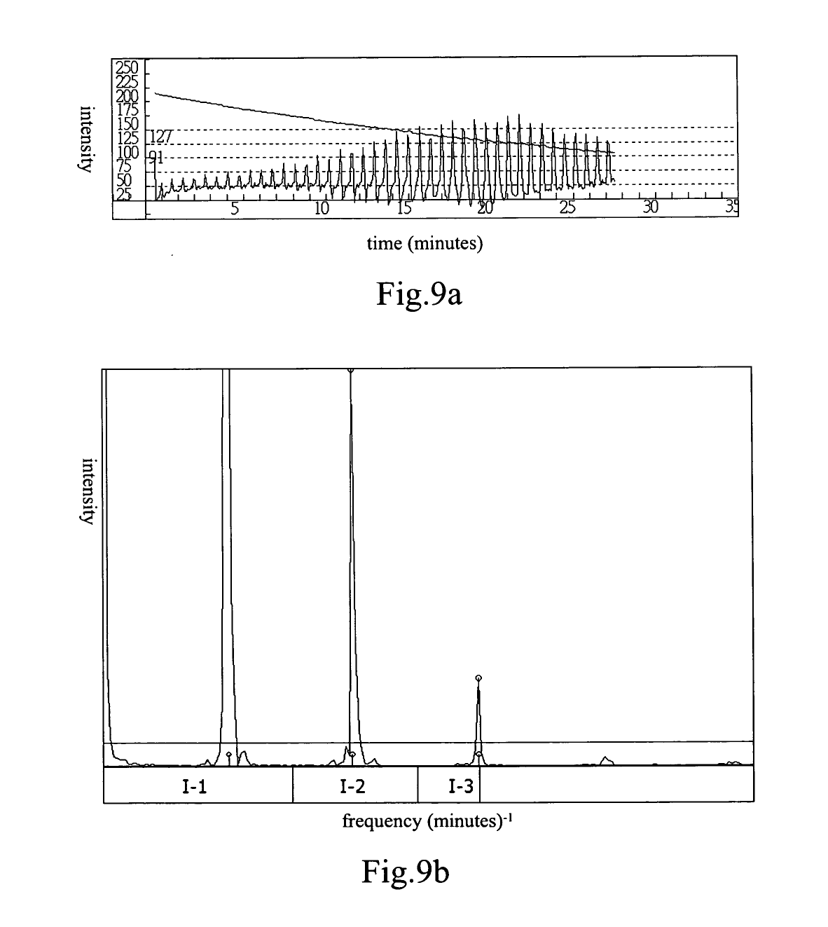

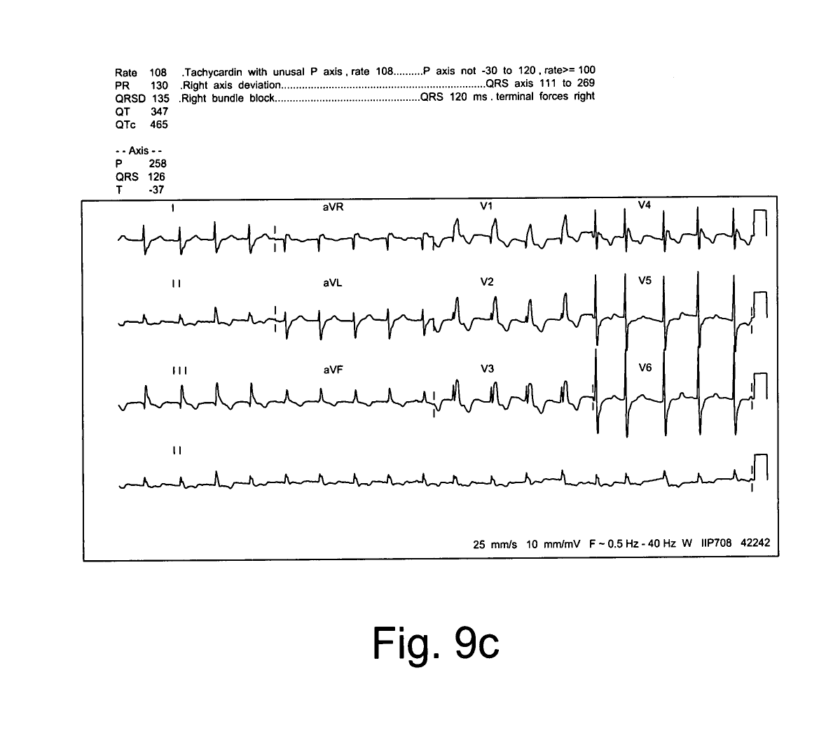

For clearly understanding the present invention, two kinds of heart rhythm analysis are provided in the following. FIG. 9a is a diagram recording the electrical heartbeat signals of a human being, FIG. 9b is its frequency spectrum, and FIG. 9c is an electrocardiogram of the same human being. Shown in the FIG. 9b, only one peak corresponding to one characteristic frequency presents within the first region, hence the heart rhythm has only single frequency and this data maybe infer that the noise figure of the heart is zero, which is in agreement with the checking result in accordance with the electrocardiogram shown in FIG. 9c. Therefore, utilizing the foregoing analysis method, the heart condition can be rapidly analyzed.

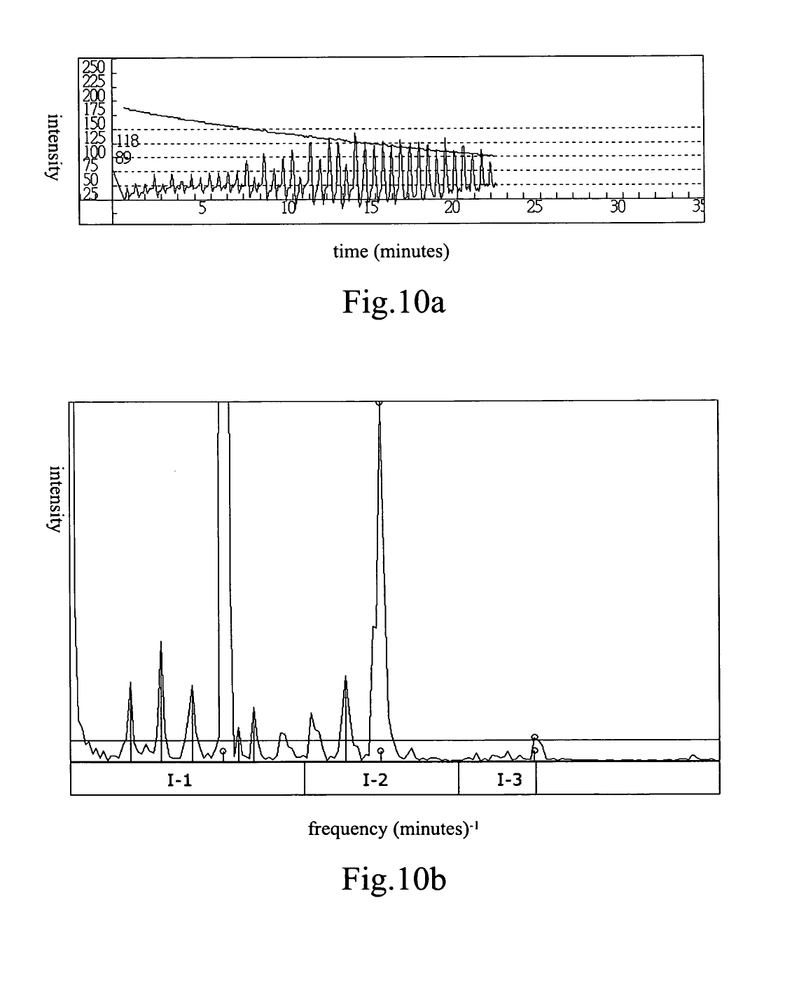

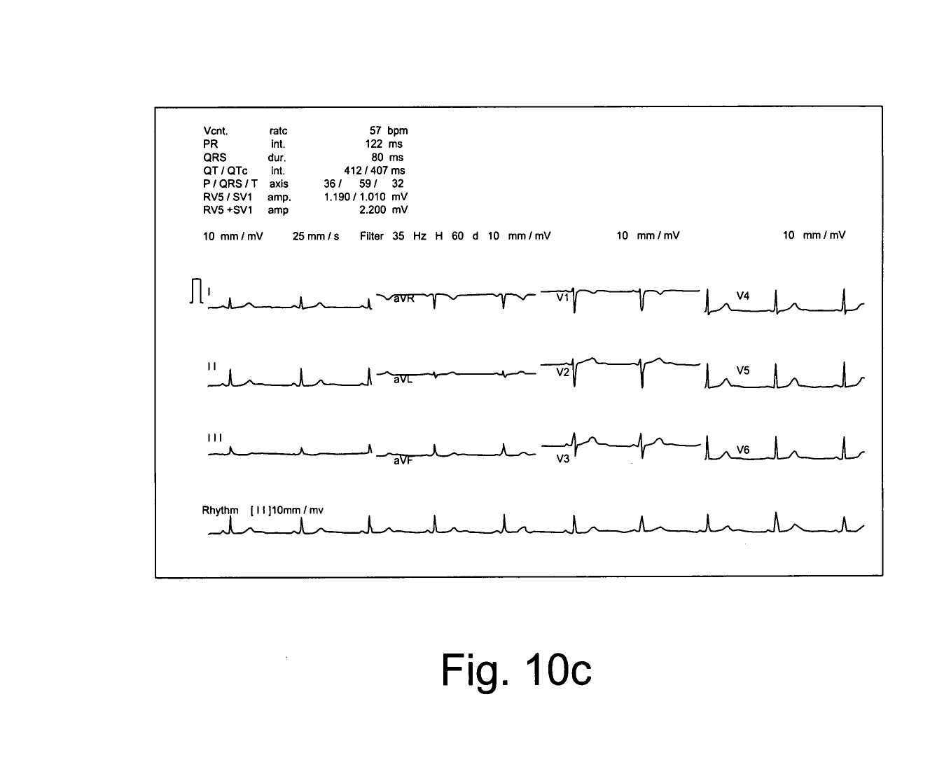

Accordingly, FIG. 10a is a diagram recording the electrical heartbeat signals of another human being, FIG. 10b is its frequency spectrum, and FIG. 10c is an electrocardiogram of the same human being. Shown in the FIG. 10b, several peaks are presented within the first region, hence the heart rhythm has several frequencies and this data maybe infer that the heart of this human being is abnormal. This inferred result is in agreement with the checking result in accordance with the electrocardiogram shown in FIG. 10c.

To sum up, the method for calculating the heart rhythm of the present invention can accurately and rapidly get the heart rhythm by the principal characteristic frequency of the frequency spectrum, no matter what the heart rhythm is too low, such as 30 times per minute, or the heart rhythm is too high, such as 240 times per minute, and so as to improve the conventional disadvantage which is unable to get too high heart rhythm or too low heart rhythm by using the sphygmomanometer. Besides, the method for analyzing the heart rhythm of the present invention accurately get the number of the heart rhythm frequency by judging the number of the characteristic frequency within the first range and roughly infer the heart condition.

Although the present invention has been explained in relation to its preferred embodiment, it is to be understood that other modifications and variation can be made without departing the spirit and scope of the invention as hereafter claimed.

What is claimed is:

1. A method for calculating heart rhythm, comprising:
measuring a plurality of electrical heartbeat signals;
evaluating said electrical heartbeat signals on the basis of a Fourier transformation to calculate a frequency spectrum of said electrical heartbeat signals, wherein at least a characteristic frequency presents within a frequency range of said frequency spectrum; and
utilizing said characteristic frequency to get said heart rhythm.

2. The method for calculating heart rhythm according to claim 1, wherein only a principal characteristic frequency presents within said frequency range, and said principal characteristic frequency corresponds to said heart rhythm.

3. The method for calculating heart rhythm according to claim 1, wherein there are a principal characteristic frequency and other characteristic frequencies within said frequency range, and said principal characteristic frequency corresponds to said heart rhythm.

4. The method for calculating heart rhythm according to claim 1, wherein said electrical heartbeat signals are measured by infrared signals, thermal radiation signals, sound signals, current signals, voltage signals, magnetic induction signals or pressure signals.

5. The method for calculating heart rhythm according to claim 1, wherein said electrical heartbeat signals are measured by a heart monitor or a physiological monitoring apparatus.

6. A method for analyzing heart rhythm, comprising:
measuring a plurality of electrical heartbeat signals;
evaluating said electrical heartbeat signals on the basis of a Fourier transformation to calculate a frequency spectrum of said electrical heartbeat signals; and
judging a number of a characteristic frequency within a frequency range of said frequency spectrum to decide a frequency number of said heart rhythm.

7. The method for analyzing heart rhythm according to claim 6, wherein said frequency range is from zero to a 1.5 times of a heartbeat number per minute.

8. The method for analyzing heart rhythm according to claim 6, wherein a principal characteristic frequency is within said frequency spectrum, and said frequency range is in the range from 0.84 times of said principal characteristic frequency to 1.16 times of said principal characteristic frequency.

9. The method for analyzing heart rhythm according to claim 7 or claim 8, wherein if only single said characteristic frequency presents within said frequency range, said heart rhythm has one frequency.

10. The method for analyzing heart rhythm according to claim 7 or claim 8, wherein if more than one said characteristic frequency presents within said frequency range, said heart rhythm has more than one frequency.

11. The method for analyzing heart rhythm according to claim 6, wherein said electrical heartbeat signals are measured by infrared signals, thermal radiation signals, sound signals, current signals, voltage signals, magnetic induction signals or pressure signals.

12. The method for analyzing heart rhythm according to claim 6, wherein said electrical heartbeat signals are measured by a heart monitor or a physiological monitoring apparatus.

13. A method for analyzing heart rhythm, comprising:
measuring a plurality of electrical heartbeat signals;
evaluating said electrical heartbeat signals on the basis of a Fourier transformation to calculate a frequency spectrum of said electrical heartbeat signals; and
counting a number of a characteristic frequency within a frequency range of said frequency spectrum to judge whether said heart rhythm is under a condition of arrhythmia.

14. The method for analyzing heart rhythm according to claim 13, wherein said frequency range is from zero to a 1.5 times of a heartbeat number per minute.

15. The method for analyzing heart rhythm according to claim 13, wherein there is a principal characteristic frequency within said frequency spectrum, and said frequency range is in the range from 0.84 times of said principal characteristic frequency to 1.16 times of said principal characteristic frequency.

16. The method for analyzing heart rhythm according to claim 14 or claim 15, wherein if only single said characteristic frequency presents within said frequency range, said heart rhythm is under a normal condition.

17. The method for analyzing heart rhythm according to claim 14 or claim 15, wherein if more than one said characteristic frequency presents within said frequency range, said heart rhythm is under a condition of arrhythmia.

18. The method for analyzing heart rhythm according to claim 13, wherein said electrical heartbeat signals are measured by infrared signals, thermal radiation signals, sound signals, current signals, voltage signals, magnetic induction signals or pressure signals.

19. The method for analyzing heart rhythm according to claim 13, wherein said electrical heartbeat signals are measured by a heart monitor or a physiological monitoring apparatus.
